(19) United States
(12) Patent Application Publication
(10) Pub. No.: US 2020/0121508 A1
(43) Pub. Date: Apr. 23, 2020

(54) FILLABLE SOFT MEDIUM EAR PROTECTOR

(71) Applicant: Pen-Li CHEN, Taipei City (TW)

(72) Inventors: Pen-Li CHEN, Taipei City (TW); Pei-Chun LI, Taipei City (TW); Chun-Wen WANG, Kaohsiung City (TW)

(21) Appl. No.: 16/205,828

(22) Filed: Nov. 30, 2018

(30) Foreign Application Priority Data
Oct. 19, 2018 (TW) 107136903

(51) Int. Cl. A61F 11/10

(52) U.S. Cl. CPC A61F 11/10; A61F 2011/085

(57) ABSTRACT

A fillable soft medium ear protector, which is used to place inside human ear canal, including a guide portion, a first fill portion disposed adjacent to the guide portion and provided with a first holding chamber, a second fill portion disposed adjacent to the first fill portion relative to the guide portion and provided with a second holding chamber, and a hollow passageway that extends between the first and the second fill portions. The hollow passageway passes through two connecting passages that respectively connect with a first and a second holding chambers. In addition, valve bodies are respectively provided between the first and the second holding chambers and the two connecting passages, thereby preventing a soft medium from leaking from the respective holding chambers. The two connecting passages enable a soft medium to respectively enter the first and the second holding chambers to form filled states therein.

### BACKGROUND OF THE INVENTION

### (a) Field of the Invention

The present invention relates to an ear protector, and more particularly to an ear protector used to place inside human ear canal and applies a filling method to provide human ear canal with a good hermetically sealed effect, as well as being further provided with high adaptability.

### (b) Description of the Prior Art

Ear protector products are mainly used for placing inside an ear canal of human being to completely cut off noise from entering the ear to achieve protection of the ear canal.

Commonly seen ear protector products mainly use an integral body formed from foam or silicone to produce a multi-disc umbrella shaped body that facilitates placement inside human ear canal to achieve a protection effect. However, after insertion into the ear canal for a long period of time, these types of ear protector products easily result in discomfort in the ear canal, and is unable to satisfy various different types of ear canals.

Hence, presently, various different inflatable ear protector products have appeared on the market, such as U.S. Publication No. 20090173353 or, U.S. Publication No. 2015282990, these types of inflatable ear protector products directly fill bags with air, however, after inflating, these types of inflatable ear protector products are unable to separately adjust the inflation state of the bags, which easily causes discomfort when wearing the ear protector.

U.S. Pat. No. 9,326,059 discloses another type of inflatable ear protector product, which is configured with a plurality of bags that are assembled and mutually connected using an encircling center method. When inflating the ear protector, the plurality of bags are concurrently inflated, however, during inflation, this type of inflatable ear protector product is similarly unable to separately adjust the inflation state of the plurality of bags, which not only easily causes discomfort when wearing the ear protector, but is also unsuitable for various different types of ear canals, and thus unable to satisfy various different ear canals, resulting in poor application effectiveness.

Accordingly, the technical problems the present invention intends to resolve and objectives thereof involves providing a ear protector having high adaptability.

### SUMMARY OF THE INVENTION

The main object of the present invention lies in providing an ear protector, and more particularly to an ear protector used to place inside an ear canal of human being and applies a filling method to provide the canal with a good hermetically sealed effect, as well as being further provided with high adaptability.

In order to achieve the aforementioned objects, a fillable soft medium ear protector of the present invention comprises a guide portion, a first fill portion, a second fill portion, and a hollow passageway. The guide portion is used to guide the ear protector into human ear canal. The first fill portion is disposed adjacent to the guide portion, and the interior of the first fill portion is provided with a first holding chamber. The second fill portion is disposed adjacent to the first fill portion relative to the guide portion, and the interior of the second fill portion is provided with a second holding chamber. The hollow passageway extends between the first fill portion and the second fill portion, and a first connecting passage that affords passage to the first holding chamber and a second connecting passage that affords passage to the second holding chamber are provided on the hollow passageway. A first valve body is disposed between the first holding chamber and the first connecting passage, and a second valve body is disposed between and the second holding chamber and the second connecting passage, thereby enabling a soft medium to pass through the first connecting passage to enter the interior of the first holding chamber and pass through the second connecting passage to enter the interior of the second holding chamber to form filled states therein. The first valve body and the second valve body are then used to respectively prevent the soft medium discharging from the first holding chamber and the second holding chamber.

In another embodiment, the hollow passageway further extends into and penetrates the guide portion, causing the hollow passageway to successively penetrate the guide portion, the first fill portion, and the second fill portion.

In the embodiment, the ear protector is further assembled from a main body and a cover body that covers the exterior of the main body. The hollow passageway, the first connecting passage, and the second connecting passage are positioned on the main body; the guide portion, the first fill portion, and the second fill portion are configured on the cover body.

In the embodiment, the first holding chamber is assembled from the first fill portion and the main body; the second holding chamber is assembled from the second fill portion and the main body.

In the embodiment, the main body and the cover body are respectively fabricated from soft material of different hardness, wherein the hardness of the main body is greater than the hardness of the cover body.

In the embodiment, the external diameter of the first fill portion is smaller than the external diameter of the second fill portion.

In the embodiment, the external diameter of the guide portion is smaller than the external diameter of the first fill portion.

In the embodiment, the main body is further provided with a first section covered by the cover body and a second section extending from the first section to the outside of the second fill portion. Accordingly, the first connecting passage and the second connecting passage are positioned on the first section, and the hollow passageway extends into the first section and the second section.

In the embodiment, the external diameter of the first section is smaller than the external diameter of the second section, causing a step portion to form between the first section and the second section.

In the embodiment, a first locating groove is further formed center of the hollow passageway to afford passage to the first connecting passage, and a second locating groove is further formed center of the hollow passageway to afford passage to the second connecting passage.

The fillable soft medium ear protector of the present invention is provided with the following advantages compared to the prior art:

The first fill portion and the second fill portion can be independently filled with the soft medium, enabling adjustment of the hard/softness within the first fill portion and second fill portion according to a variety of ear canals, further achieving a significantly better airtight effect and wider applicability, as well as effectively achieving a more protective effect for human ear canal.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 is a three-dimensional schematic view of the first embodiment of the present invention.

FIG. 2 is a first cross-sectional schematic view of the first embodiment of the present invention.

FIG. 3 is a partial cross-sectional schematic view of the first embodiment of the present invention.

FIG. 4 is a second cross-sectional schematic view of the first embodiment of the present invention.

FIG. 5 is an exploded three-dimensional schematic view of the first embodiment of the present invention.

FIG. 6 is a cross-sectional schematic view of the main body of the present invention.

FIG. 7 is a cross-sectional schematic view of a cover body of the present invention.

FIG. 8 is a reference diagram of an operating state of the first embodiment of the present invention.

FIG. 9 is a three-dimensional schematic view of a second embodiment of the present invention.

FIG. 10 is a cross-sectional schematic view of the second embodiment of the present invention.

### DETAILED DESCRIPTION OF THE PREFERRED EMBODIMENTS

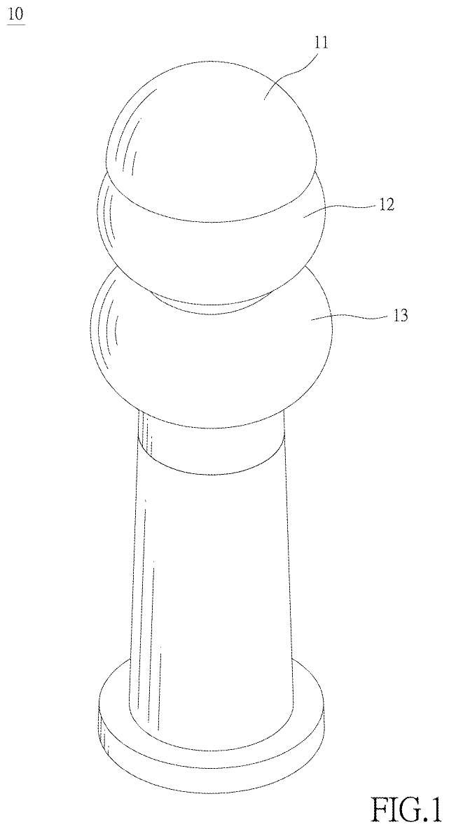

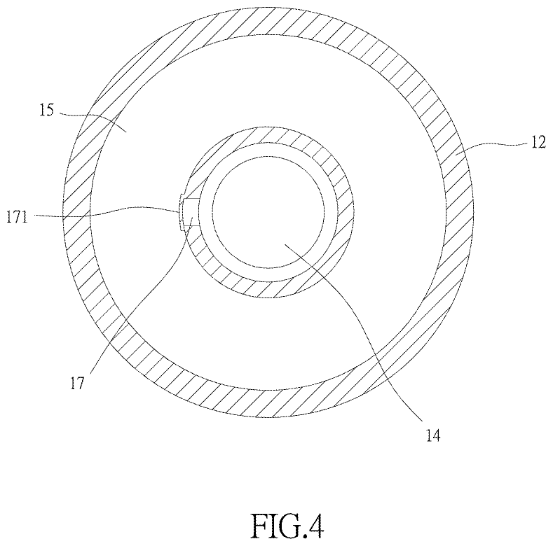

Regarding the detailed content and structural means of the present invention, please refer to the description of the diagrams as follows: Referring to FIGS. 1 to 4, the present invention provides a fillable soft medium ear protector, wherein an ear protector 10 comprises a guide portion 11, a first fill portion 12, a second fill portion 13, and a hollow passageway 14. The guide portion 11 is used to guide the ear protector 10 into an ear canal of human being (not shown in the drawings). The first fill portion 12 is disposed adjacent to the guide portion 11, with the external diameter of the guide portion 11 being smaller than the external diameter of the first fill portion 12, and the interior of the first fill portion 12 is provided with a first holding chamber 15. The second fill portion 13 is adjacent to the first fill portion 12 and disposed relative to the guide portion 11, thereby causing the guide portion 11 and the second fill portion 13 to be positioned on two corresponding sides of the first fill portion 12. Moreover, the external diameter of the first fill portion 12 is smaller than the external diameter of the second fill portion 13, causing the external diameters of the assembled guide portion 11, the first fill portion 12, and the second fill portion 13 to form a pyramidal shape. The interior of the second fill portion 13 is provided with a second holding chamber 16, and the hollow passageway 14 extends between the first fill portion 12 and the second fill portion 13. The first holding chamber 15 and the second holding chamber 16 respectively encircle the hollow passageway 14 and are mutually separated therefrom, as shown in FIG. 4. A first connecting passage 17 and a second connecting passage 18 are respectively separated and provided on the hollow passageway 14, enabling the first holding chamber 15 to connect with the hollow passageway 14 through the first connecting passage 17, and enabling the second holding chamber 16 to connect with the hollow passageway 14 through the second connecting passage 18, thereby enabling a soft medium (not shown in the drawings, but depicted as arrowheads to indicate the flowing motion thereof, with the soft medium being a gas or liquid provided with fluidity), after passing through the hollow passageway 14, to respectively enter the interior of the first holding chamber 15 by passing through the first connecting passage 17 and to enter the interior of the second holding chamber 16 by passing through the second connecting passage 18, thereby causing the first fill portion 12 and the second fill portion 13 to form filled states.

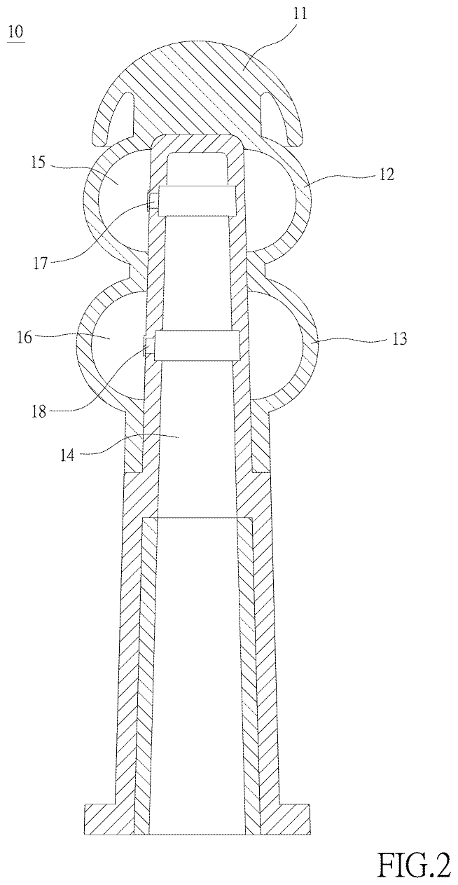

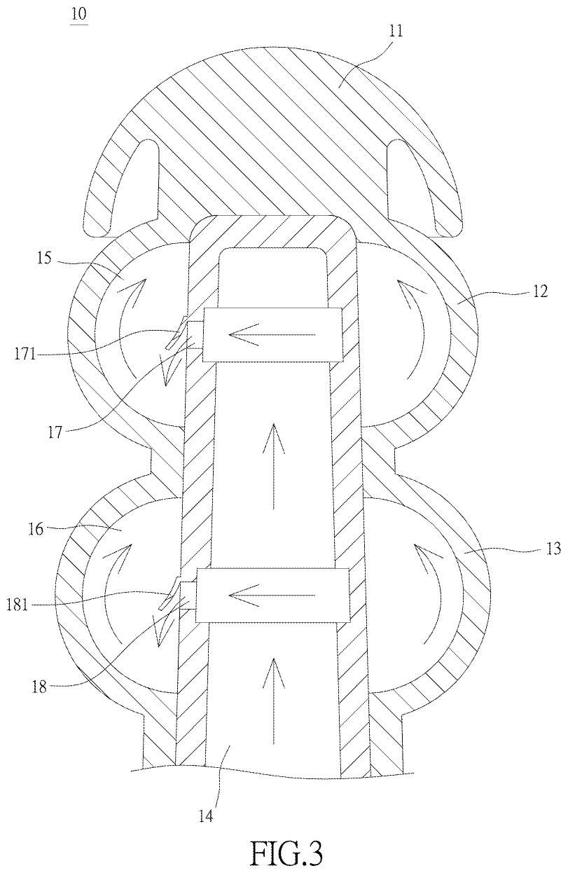

Furthermore, in order to avoid the first fill portion 12 and the second fill portion 13 maintaining filled states, a first valve body 171 is disposed between the first holding chamber 15 and the first connecting passage 17, and a second valve body 181 is disposed between the second holding chamber 16 and the second connecting passage 18. In the embodiment, the first valve body 171 and the second valve body 181 are both valve plates and cover the external portions of the first connecting passage 17 and the second connecting passage 18, respectively, to form cut-off valves that are used to stop outward discharge of the soft medium from the first holding chamber 15 and second holding chamber 16. Accordingly, when the soft medium respectively passes through the first connecting passage 17 and the second connecting passage 18, air pressure of the soft medium pushes the first valve body 171 and the second valve body 181 toward the interior of the first holding chamber 15 and second holding chamber 16, respectively, causing portions of the first valve body 171 and second valve body 181 to separate from the first connecting passage 17 and the second connecting passage 18, further enabling the soft medium to smoothly enter the interior of the first holding chamber 15 and the second holding chamber 16, respectively, as shown in FIG. 3. When the soft medium stops passing through the first connecting passage 17 and second connecting passage 18, the pressure inside the first holding chamber 15 and second holding chamber 16 is greater than the pressure inside the first connecting passage 17 and second connecting passage 18, respectively, at which time, the soft medium positioned inside the first holding chamber 15 and second holding chamber 16 respectively pushes against the first valve body 171 and second valve body 181, thereby covering the first connecting passage 17 and second connecting passage 18, respectively, thus, effectively preventing the soft medium from discharging from the interior of the first holding chamber 15 and second holding chamber 16 and enabling the first fill portion 12 and the second fill portion 13 to effectively maintain filled states, as shown in FIG. 2.

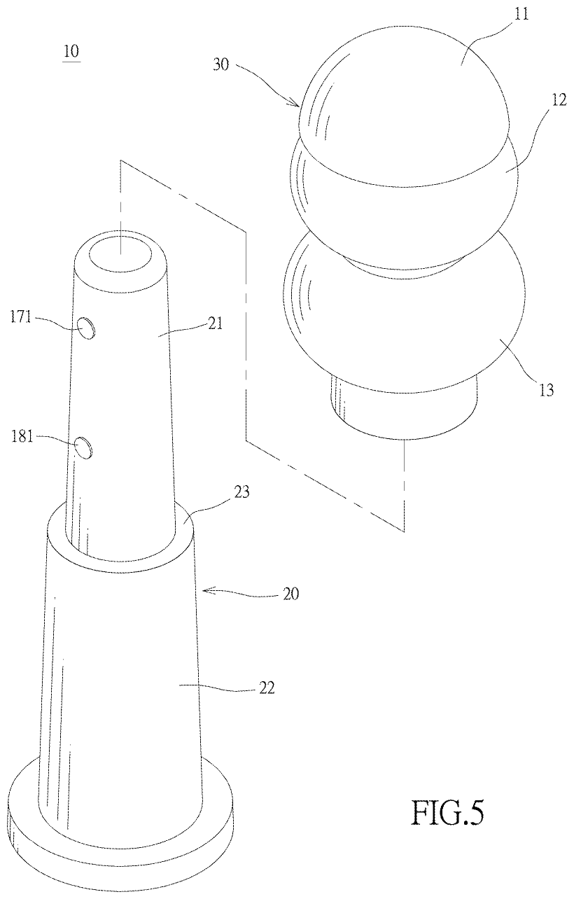

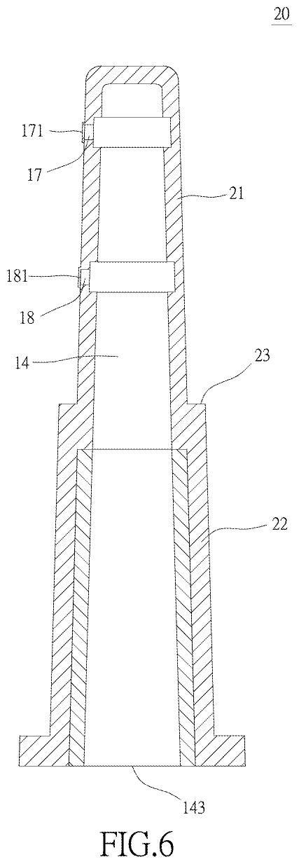

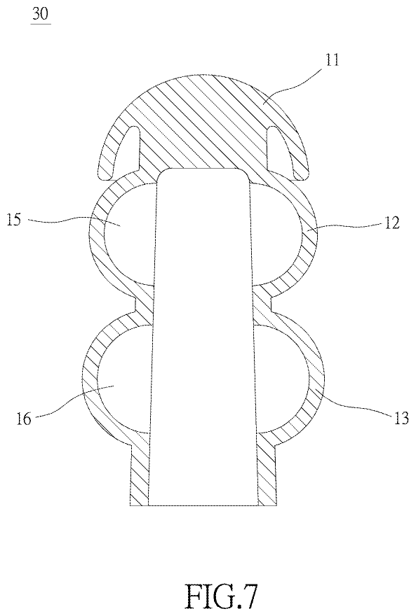

Referring to FIGS. 5 to 7, in order to facilitate production, the ear protector 10 in the present embodiment is mainly assembled from a main body 20 and a cover body 30 covering the exterior of the main body 20, wherein the main body 20 is provided with a first section 21 covered by the cover body 30 and a second section 22 extending from the first section 21 to the outside of the second fill portion 13. Moreover, the external diameter of the first section 21 is smaller than the external diameter of the second section 22, causing a step portion 23 to form between the first section 21 and the second section 22. The hollow passageway 14 extends into the interior of the first section 21 and the second section 22, and an opening 143 is formed at an end portion of the second section 22 distant from the first section 21. Hence, the first connecting passage 17 and the second connecting passage 18 are provided on the first section 21, as shown in FIG. 6. In addition, the first valve body 171 and the second valve body 181 in the present embodiment are structured to form single bodies on the main body 20, with a part of each of the first valve body 171 and the second valve body 181 is connected to the first section 21 while another part is able to separate from the main body 20, enabling the first valve body 171 and the second valve body 181 to achieve the effectiveness of cut-off valves as described above and shown in FIG. 3.

The cover body 30 is used to cover the exterior of the first section 21, and the guide portion 11, the first fill portion 12, and the second fill portion 13 are configured on the cover body 30. The first holding chamber 15 is structured and formed from the first fill portion 12 and the first section 21 of the main body 20, and the second holding chamber 16 is structured and formed from the second fill portion 13 and the first section 21 of the main body 20, causing mutual separation between the first holding chamber 15 and the second holding chamber 16.

Furthermore, the main body 20 and the cover body 30 in the present embodiment are respectively fabricated from soft material of different hardness, such as silicone, etc. The hardness of the main body 20 is greater than the hardness of the cover body 30, enabling the ear protector 10 to more effectively improve wearing comfort thereof when placed into human ear canal. In addition, the hard/softness inside the first holding chamber 15 and the second holding chamber 16 within the first fill portion 12 and the second fill portion 13 can be adjusted in accordance with different sizes of human ear canal, thereby enabling the ear protector 10 to be suitable for use in a variety of ear canals, thus, increasing applicability of the ear protector 10. Furthermore, adjustment of the hard/softness inside the first holding chamber 15 and the second holding chamber 16 enable the first fill portion 12 and the second fill portion 13 to better fit the inner wall of the ear canal of human being, further improving airtightness inside the ear canal, and, thus, achieving a more favorable protective effect for the ear canal.

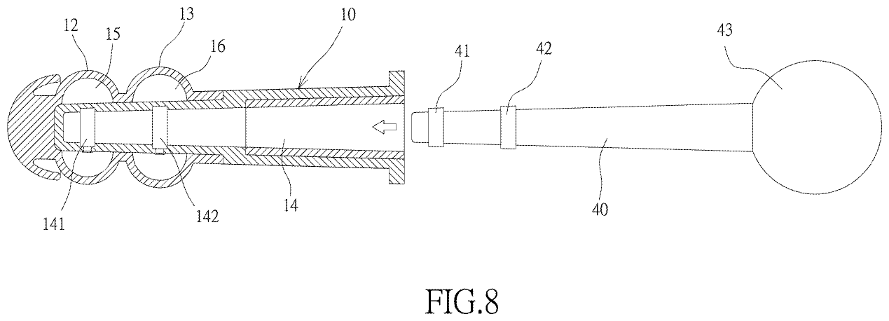

Referring to FIG. 2 and FIGS. 6 to 8, when using the ear protector 10 of the present invention, the basic principle is to insert a fill member 40 inside the hollow passageway 14, and use the fill member 40 to supply a soft medium into the first fill portion 12 and the second fill portion 13 to become filled states. A first supply portion 41 corresponding to the first connecting passage 17, a second supply portion 42 corresponding to the second connecting passage 18, and an air bag portion 43 used to supply the soft medium are formed on the fill member 40. After the fill member 40 is inserted inside the ear protector 10 through the hollow passageway 14, the first supply portion 41 is positioned corresponding to the first connecting passage 17 and the second supply portion 42 is positioned corresponding to the second connecting passage 18, the air bag portion 43 is then pressed to respectively channel a soft medium into the interior of the first connecting passage 17 and the second connecting passage 18, thereafter further entering the interior of the first holding chamber 15 and the second holding chamber 16, enabling the first fill portion 12 and the second fill portion 13 to become filled states. In order to facilitate the first fill portion 12 and the second fill portion 13 to precisely correspond to the positions of the first connecting passage 17 and the second connecting passage 18, a first locating groove 141 is further formed center of the hollow passageway 14 corresponding to the position of the first connecting passage 17 to enable fixed positioning of the first supply portion 41, and a second locating groove 142 is further formed center of the hollow passageway 14 corresponding to the position of the second connecting passage 18 to enable fixed positioning of the second supply portion 42, thereby effectively achieving fixed positioning of the fill member 40 inside the hollow passageway 14. However, although, the fill member 40 in the present embodiment concurrently inflates the first fill portion 12 and the second fill portion 13, during actual use, the fill member 40 is also able to individually inflate the first fill portion 12 or the second fill portion 13, thereby achieving single filling effectiveness of the first fill portion 12 or the second fill portion 13, and further enabling separate adjustment of the hard/softness within the first fill portion 12 or second fill portion 13 to improve applicability of the ear protector 10.

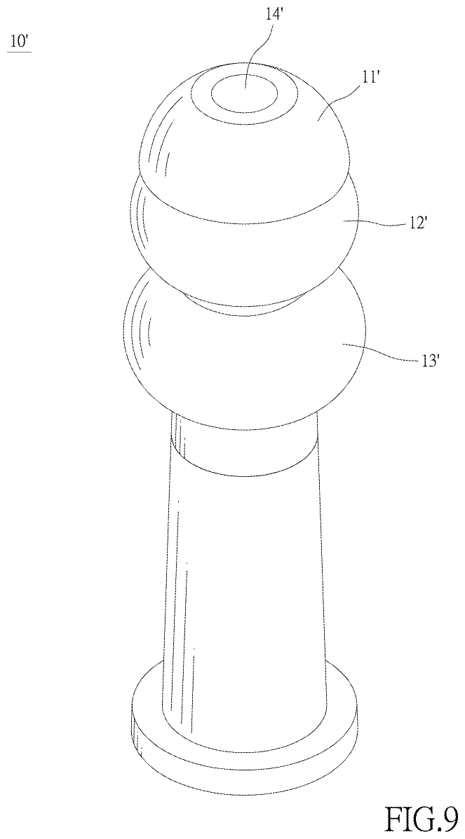

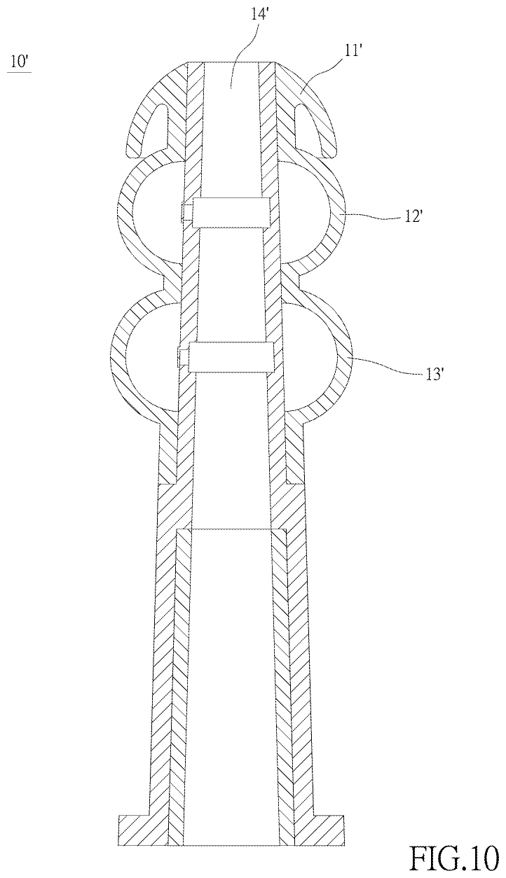

Further referring to FIGS. 9 and 10, which show another embodiment of the present embodiment, wherein a hollow passageway 14′ further extends into and penetrates a guide portion 11′, causing the hollow passageway 14′ to successively penetrate the guide portion 11′, a first fill portion 12′, and a second fill portion 13′. Hence, when an ear protector 10′ is placed inside human ear canal, external air is able to pass through the hollow passageway 14′ and circulate inside the ear canal. Accordingly, apart from preventing excessive air pressure inside the ear canal, enabling external air to pass through the hollow passageway 14′ also enables the ear canal to receive external sounds, further improving safety of the ear protector 10′ when is use.

In conclusion, the present invention clearly complies with the essential elements as required for a new invention patent, thus, a new patent application is proposed herein. It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A fillable soft medium ear protector, comprising:
a guide portion, which is used for guiding into an ear canal of human being;
a first fill portion, which is disposed adjacent to the guide portion, an interior of the first fill portion is provided with a first holding chamber;
a second fill portion, which is adjacent to the first fill portion and disposed correspondingly to the guide portion, an interior of the second fill portion is provided with a second holding chamber; and
a hollow passageway, which extends between the first fill portion and the second fill portion, the hollow passageway is provided with a first connecting passage that affords passage to the first holding chamber and a second connecting passage that affords passage to the second holding chamber, a first valve body is provided between the first holding chamber and the first connecting passage, and a second valve body is provided between the second holding chamber and the second connecting passage, thereby enabling a soft medium to enter the interior of the first holding chamber and the second holding chamber to form filled states through the first connecting passage and the second connecting passage, respectively; moreover, the first valve body and the second valve body are used to prevent the soft medium discharging from the first holding chamber and the second holding chamber, respectively.

2. The fillable soft medium ear protector according to claim 1, wherein the hollow passageway further extends into and penetrates the guide portion, causing the hollow passageway to successively penetrate the guide portion, the first fill portion, and the second fill portion.

3. The fillable soft medium ear protector according to claim 1, wherein the ear protector is assembled from a main body and a cover body that covers an exterior of the main body; the hollow passageway, the first connecting passage, and the second connecting passage are positioned on the main body, and the guide portion, the first fill portion, and the second fill portion are configured on the cover body.

4. The fillable soft medium ear protector according to claim 2, wherein the ear protector is assembled from a main body and a cover body that covers an exterior of the main body; the hollow passageway, the first connecting passage, and the second connecting passage are positioned on the main body, and the guide portion, the first fill portion, and the second fill portion are configured on the cover body.

5. The fillable soft medium ear protector according to claim 3, wherein the first holding chamber is assembled from the first fill portion and the main body; the second holding chamber is assembled from the second fill portion and the main body.

6. The fillable soft medium ear protector according to claim 4, wherein the first holding chamber is assembled from the first fill portion and the main body; the second holding chamber is assembled from the second fill portion and the main body.

7. The fillable soft medium ear protector according to claim 3, wherein the main body and the cover body are respectively fabricated from soft material of different hardness, wherein the hardness of the main body is greater than the hardness of the cover body.

8. The fillable soft medium ear protector according to claim 4, wherein the main body and the cover body are respectively fabricated from soft material of different hardness, wherein the hardness of the main body is greater than the hardness of the cover body.

9. The fillable soft medium ear protector according to claim 3, wherein the external diameter of the first fill portion is smaller than an external diameter of the second fill portion.

10. The fillable soft medium ear protector according to claim 4, wherein the external diameter of the first fill portion is smaller than an external diameter of the second fill portion.

11. The fillable soft medium ear protector according to claim 9, wherein the external diameter of the guide portion is smaller than an external diameter of the first fill portion.

12. The fillable soft medium ear protector according to claim 10, wherein the external diameter of the guide portion is smaller than an external diameter of the first fill portion.

13. The fillable soft medium ear protector according to claim 3, wherein the main body is further provided with a first section covered by the cover body and a second section extending from the first section to an outside of the second fill portion; the first connecting passage and the second connecting passage are positioned on the first section, and the hollow passageway extends into the first section and the second section.

14. The fillable soft medium ear protector according to claim 4, wherein the main body is further provided with a first section covered by the cover body and a second section extending from the first section to an outside of the second fill portion; the first connecting passage and the second connecting passage are positioned on the first section, and the hollow passageway extends into the first section and the second section.

15. The fillable soft medium ear protector according to claim 13, wherein an external diameter of the first section is smaller than an external diameter of the second section, causing a step portion to form between the first section and the second section.

16. The fillable soft medium ear protector according to claim 14, wherein an external diameter of the first section is smaller than an external diameter of the second section, causing a step portion to form between the first section and the second section.

17. The fillable soft medium ear protector according to claim 1, wherein a first locating groove is further formed at a location center of the hollow passageway to afford passage to the first connecting passage, and a second locating groove is further formed at a location center of the hollow passageway to afford passage to the second connecting passage.
